(19) United States
(12) Patent Application Publication
(10) Pub. No.: US 2019/0046061 A1
(43) Pub. Date: Feb. 14, 2019

(54) COMPUTING DEVICE WITH MULTI-PART ELECTRODES

(71) Applicant: Intel Corporation, Santa Clara, CA (US)

(72) Inventors: Sukanya Sundaresan, Bangalore (IN); Ayeshwarya B. Mahajan, Bangalore (IN)

(73) Assignee: Intel Corporation, Santa Clara, CA (US)

(21) Appl. No.: 16/041,447

(22) Filed: Jul. 20, 2018

(51) Int. Cl. A61B 5/0408; A61B 5/04; A61B 5/00

(52) U.S. Cl. CPC A61B 5/0408; A61B 5/04012; A61B 2560/0468; A61B 2560/0406; A61B 5/0006

(57) ABSTRACT

In embodiments, a chassis of a computing device may include an external surface that is accessible by a user of the computing device, and an internal surface opposite the external surface. The chassis may further include a first electrode and a second electrode within the external surface. The first electrode may generate a first signal related to a first biosignal of the user of the computing device, and the second electrode may generate a second signal related to a second biosignal of the user. Other embodiments may be described and/or claimed.

### BACKGROUND

Consumer grade biosensing may enable multiple usages for wellness, fitness, or emotion sensing applications. Biosensors may be integrated into a dedicated device like a fitness watch, or integrated into consumer devices like mobile phones.

### BRIEF DESCRIPTION OF THE DRAWINGS

FIG. 1 depicts one example of a computing device that may include a plurality of electrodes, in accordance with various embodiments.

FIG. 2 depicts an example of a technique for generating a chassis that includes a plurality of electrodes, in accordance with various embodiments.

FIG. 3 further depicts an example of the technique for generating a chassis that includes a plurality of electrodes, in accordance with various embodiments.

FIG. 4 depicts an alternative embodiment of a chassis, in accordance with various embodiments.

FIG. 5 depicts a simplified cross-sectional view of an electrode, in accordance with various embodiments.

FIG. 6 depicts an example technique by which a chassis with one or more electrodes and an electrical interconnect may be formed, in accordance with various embodiments.

FIG. 7 illustrates an example device, in accordance with various embodiments.

### DETAILED DESCRIPTION

In the following detailed description, reference is made to the accompanying drawings which form a part hereof, wherein like numerals designate like parts throughout, and in which is shown by way of illustration embodiments in which the subject matter of the present disclosure may be practiced. It is to be understood that other embodiments may be utilized and structural or logical changes may be made without departing from the scope of the present disclosure. Therefore, the following detailed description is not to be taken in a limiting sense.

For the purposes of the present disclosure, the phrase “A or B” means (A), (B), or (A and B). For the purposes of the present disclosure, the phrase “A, B, or C” means (A), (B), (C), (A and B), (A and C), (B and C), or (A, B and C).

The description may use perspective-based descriptions such as top/bottom, in/out, over/under, and the like. Such descriptions are merely used to facilitate the discussion and are not intended to restrict the application of embodiments described herein to any particular orientation.

The description may use the phrases “in an embodiment,” or “in embodiments,” which may each refer to one or more of the same or different embodiments. Furthermore, the terms “comprising,” “including,” “having,” and the like, as used with respect to embodiments of the present disclosure, are synonymous.

The term “coupled with,” along with its derivatives, may be used herein. “Coupled” may mean one or more of the following. “Coupled” may mean that two or more elements are in direct physical or electrical contact. However, “coupled” may also mean that two or more elements indirectly contact each other, but yet still cooperate or interact with each other, and may mean that one or more other elements are coupled or connected between the elements that are said to be coupled with each other. The term “directly coupled” may mean that two or elements are in direct contact.

In various embodiments, the phrase “a first feature formed, deposited, or otherwise disposed on a second feature,” may mean that the first feature is formed, deposited, or disposed over the feature layer, and at least a part of the first feature may be in direct contact (e.g., direct physical or electrical contact) or indirect contact (e.g., having one or more other features between the first feature and the second feature) with at least a part of the second feature.

Various operations may be described as multiple discrete operations in turn, in a manner that is most helpful in understanding the claimed subject matter. However, the order of description should not be construed as to imply that these operations are necessarily order dependent.

Embodiments herein may be described with respect to various Figures. Unless explicitly stated, the dimensions of the Figures are intended to be simplified illustrative examples, rather than depictions of relative dimensions. For example, various lengths/widths/heights of elements in the Figures may not be drawn to scale unless indicated otherwise.

Legacy contact-based biosensors may have electrodes that take the form of metal plates, a conductive fabric, a conductive foam, etc. However, these legacy electrodes may have included one or more drawbacks based on their reliance on a single, unbroken or continuous surface of sensing material that is to come in contact with the skin of a user.

One drawback may have been that the region covered by sensing material may be dedicated for one particular biosignal that is to be sensed at a particular time. This approach may prohibit simultaneous sensing of multiple biosignals. Such an example may be if the sensing material is implemented on a phone, a watch, or another computing device with a relatively small form factor.

Another drawback may have been that in many legacy devices the sensing material may be a flat metal plate which may be relatively stiff, and therefore not easily conformable to the skin of a user. As a result, reliable skin contact may not be achieved between the plate and the user, especially when the user or the device are in motion.

Another drawback may relate to industrial design limitations. Specifically, a single large continuous sensing surface (e.g., a metal plate) may negatively impact the industrial design of a computing device in which the metal plate is implemented. For example, if a metal plate is embedded in a palm rest area of a computing device, the overall design aesthetic of the device may be significantly compromised due to differences in material, color and finish between the device and the electrode, and the need to maintain electrical isolation between sensing electrodes.

By contrast, embodiments herein may incorporate multiple relatively small electrodes across the sensing surface of a computing device. The electrodes may make contact with the skin of a user on one side of the sensing surface, and may be electrically coupled with an electrical interconnect on another side of the sensing surface. Multiple such electrodes may be coupled with one another by the electrical interconnect, and may then be further coupled with one or more processors of the computing device which are able to extract, based on signals sent by the electrodes and any related conditioning electronics, measurements related to one or more biosignals of the user.

In embodiments, the electrodes may provide an aesthetic effect like speckle, or a subtle texture on the sensing surface without interfering with the overall color, material, or finish of the computing device as dictated by industrial design requirements. The electrodes may also have surface treatments like conductive hard coating that may provide multiple color options to further enhance the aesthetics of the computing device. In addition, plating processes on a core material can also provide multiple color and finish options as suited for the industrial design.

These embodiments may provide several advantages over legacy devices. Specifically, the use of multiple relatively small electrodes may provide a conformable sensing surface that allows for increased contact with the skin of the user. This increased contact may be achieved without the electrodes providing an abrasive feeling to the user or otherwise being obtrusive.

Additionally, the use of multiple relatively small biosensors may enable the sensing of multiple biosignals from generally the same region at the same time. Specifically, the conductors on the same surface may be connected differently to enable sensing of different parameters simultaneously. This may enable sensing of multiple biosignals when a user is normally using the computing device.

Additionally, as discussed above, in embodiments the multiple relatively small electrodes may enable a relatively unobtrusive industrial design. Specifically, the small biosensors, while being functional, may be almost imperceptible and blend into the chassis of the computing device without affect to color or finish of the chassis. Alternatively, the biosensors may provide a subtle effect and texture, like speckle, or offer color options through conductive surface treatment or plating processes. Finally, the multiple relatively small electrodes may enable a relatively low-cost manufacturing technique by which multiple electrodes may be provided in a computing device.

Generally, as used herein the term “electrode” may refer to a sensing surface that is able to generate a signal related to one or more biosignals of a user of a computing device in which the electrode is present. In embodiments, the electrode may also be referred to as a “biosensor.” Such a biosignal may include, for example, a measurement of skin conductivity which may be referred to as “galvanic skin response” (GSR). This measurement may relate to how well an electrical current flows through the skin and may be indicative of a user's emotional state. Another such biosignal may be related to electrocardiogram (ECG). This measurement may relate to heart-rate and be indicative of an excitement or stress level of a user. Another such biosignal may be related to skin temperature. This measurement may be indicative of a user's bloodflow which may also relate to things like stress, excitement, etc. It will be understood that these specific biosignals are intended as examples of various biosignals that may be produced based on contact with a user's skin, and other biosignals like electromyography (EMG), electroencephalogram (EEG), etc., may be present in other embodiments.

Generally, the electrode may be configured to generate one or more signals related to a biosignal of a user of the computing device. The signal may be then processed by one or more processors to generate a measurement related to the biosignal. For example, a plurality of signals related to ECG may be processed to identify a heart-rate of a user. In some embodiments the processor may be an element of the electrode itself, while in other embodiments the processor may be communicatively coupled with the electrode.

In some embodiments, a single electrode may be configured to generate signals related to two separate biosignals. Specifically, a single electrode may be configured to generate a first signal related to a first biosignal at a first time, and then a second signal related to a second biosignal at a second time. The first signal and the second signal may be transmitted to a processor where the signals are then separately processed to identify a first measurement related to the first biosignal and a second measurement related to the second biosignal. In other embodiments, the processor may be configured to identify a first measurement related to a first biosignal and a second measurement related to a second biosignal based on a single signal generated by the electrode.

In some embodiments, it may not be known which of the electrodes in a plurality of electrodes a user is in contact with. Therefore, each of the electrodes in the plurality of electrodes may produce a signal. Each of the produced signals may be analyzed by a processor to identify one or more of the signals that correspond to contact with the user. The identified signals may then be processed further to produce one or more measurements related to one or more biosignals.

It will be understood that the above descriptions are intended as examples of various embodiments, and other embodiments may include variations on these examples. For example, in some embodiments the separate signals may be concurrent in time but separated in the frequency domain. In other embodiments a third biosignal or a third signal may be generated or processed, etc.

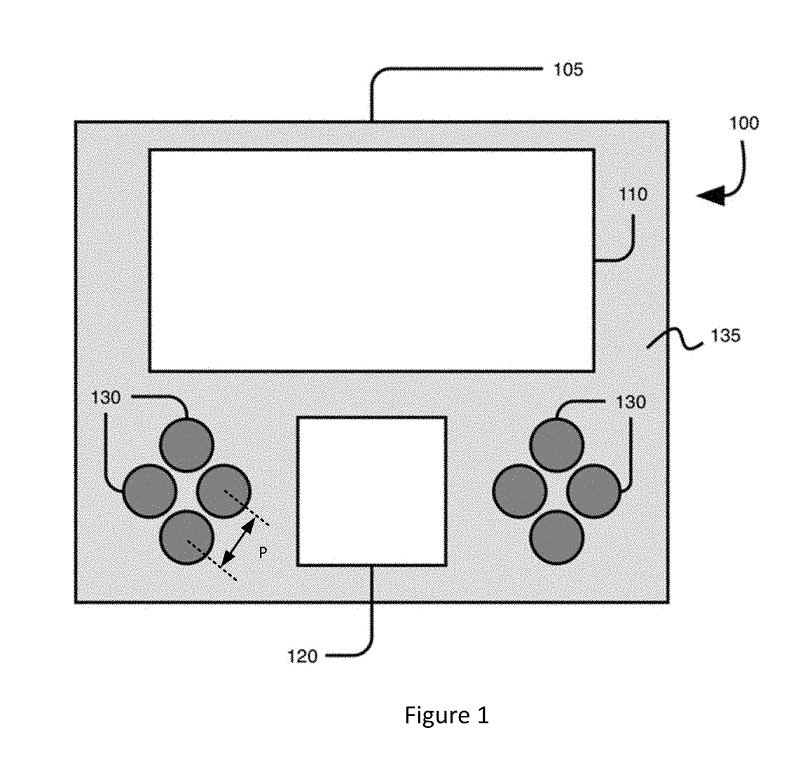

FIG. 1 depicts one example of a computing device 100 that may include a plurality of electrodes 130, in accordance with various embodiments. In this specific embodiment, the computing device 100 may be a laptop computer, however in other embodiments the computing device may be a variety of other structures in which measurements of a user's biosignals may be desirable. For example, in embodiments the computing device may be a device such as a mobile phone, a keyboard for a desktop computer or some other type of keyboard, a musical instrument such as a midi device, a remote control, a handle of a piece of sports equipment such as a bicycle, a handle of a piece of motorized equipment such as a car steering wheel or motorcycle handlebars, into portions of keycaps (like the bumps on F and J keys of a computing device), etc. In each of these computing devices electrodes such as electrodes 130 may be placed in a location in which a user may naturally interact with the electrodes, thereby allowing for efficient measurements of a user's biosignals.

The computing device 100 may have a chassis 105. The chassis 105 may be, for example, made of plastic, stainless steel, wood, anodized aluminum, a thermoplastic polymer such as acrylonitrile butadiene styrene (ABS), etc. Generally speaking, the chassis 105 may be formed of an insulating or non-conducting material that may electrically insulate the electrodes 130 from one another. In other embodiments, the chassis 105 may be formed of a conductive material but may include other elements that electrically insulate the electrodes 130 from one another. As shown in FIG. 1, the chassis 105 may generally be a base section of a laptop computer that may include a number of user interface elements as described below. However, in other embodiments the chassis 105 may be some other element of a computing device. For example, if the electrodes 130 are in the keycaps as described above, the chassis 105 may be a single keycap or a plurality of keycaps. If the electrodes 130 are positioned on a steering wheel of a car, the chassis 105 may be the steering wheel or a portion thereof. Generally, the chassis 105 may be considered to be the element of the computing device in which the electrodes 130 are embedded.

The chassis 105 may include a number of user interface elements such as user interface 110 and user interface 120. User interface element 110 may be, for example, a keyboard and user interface element 120 may be, for example, a trackpad. However, in other embodiments the chassis 105 may include additional user interface elements, or only a single user interface element. In some embodiments the user interface elements 110 or 120 may be alternative user interfaces such as a touchscreen, a gesture control, a designpad configured to interact with a stylus, a display screen, a speaker, or some other type of user interface that is able to provide information to, or receive directions from, a user of the computing device 100.

The computing device may also include a plurality of electrodes 130. As shown in FIG. 1, the electrodes 130 may be positioned on opposite sides of the chassis 105 where a user may normally rest their hands if they were to operate user interface element 110. Such a region may be referred to as, e.g., a palmrest. As shown in FIG. 1, respective ones of the electrodes 130 may have a generally circular cross-section. In the embodiment shown in FIG. 1 an electrode 130 may be a conductive microsphere that protrudes from the palm rest surface. The number of spheres, projection height and the diameter of the protrusion ensure that the overall surface area requirement is met when the user's skin makes contact with the electrodes. In a specific embodiment the electrode 130 may have a diameter of approximately 0.5 millimeters (mm). The electrode elements 130 may protrude from the external surface 135 of the chassis 105 by between approximately 0 micrometers (“microns” or “μm”) and approximately 500 microns. In a specific embodiment, the electrode element may protrude from the surface of the chassis 105 by approximately 100 microns. In some embodiments, the protrusion and its diameter may result in a total skin-contact area of at least 20 square millimeters (mm²).

As discussed above, it may be desirable for a user to contact a plurality of the electrodes 130 simultaneously. For example, respective ones of the electrodes 130 may be suited to generate a signal related to a biosignal. However, it may be desirable to take measurements related to a plurality of biosignals simultaneously. Therefore, a first electrode 130 may generate a signal that relates to an ECG. A second electrode 130 may generate a signal that relates to a skin conductivity. A third electrode 130 may generate a signal that relates to skin temperature. Because respective electrodes 130 may generate a signal related to a different biosignal, it may be desirable for the user to contact a plurality of the electrodes 130 so that each of the biosignals can be measured generally simultaneously. In some embodiments the electrodes 130 may have a pitch P of approximately 3.5 mm. More generally, the electrodes 130 may have a pitch between approximately 2 mm and approximately 5 mm. As used herein, the term “pitch” refers to a distance between the center of one of the electrodes 130 and another of the electrodes 130.

It will be understood that the specific arrangement depicted in FIG. 1 is intended as one example arrangement and other arrangements may be present in other embodiments. For example, although the electrodes 130 are depicted as arranged into two arrays of four electrodes 130 each, in other embodiments the computing device 100 may have more or fewer electrodes. In some embodiments, the computing device 100 may have two arrays of between 20 and 30 electrodes. In other embodiments the computing device 100 may only have a single array of three electrodes. In some embodiments the computing device 100 may have as many as 350 or more electrodes. Generally, the number of electrodes may be dependent on factors such as the number of biosignals being measured, the sort of computing device (a wearable such as a watch as opposed to a laptop computer or a piece of sports equipment), or some other factor.

Additionally, although FIG. 1 depicts the electrodes 130 arranged in a generally diamond shaped pattern, in other embodiments the electrodes may be arranged in a grid-like pattern, a star shaped pattern, a pattern that resembles a logo of a manufacturer of the computing device 100, etc.

Additionally, the electrodes 130 are depicted as having a generally circular cross-section and being spheres. However, in other embodiments the electrodes may have a different cross-section such as rectangular, oblong, square shaped, triangular, hexagonal, etc. Additionally, as will be discussed in further detail below, in some embodiments the electrodes may not protrude from the chassis 105, but rather the electrodes may be flush with the external surface 135 of the chassis 105. Other variations of arrangements may be present in other embodiments.

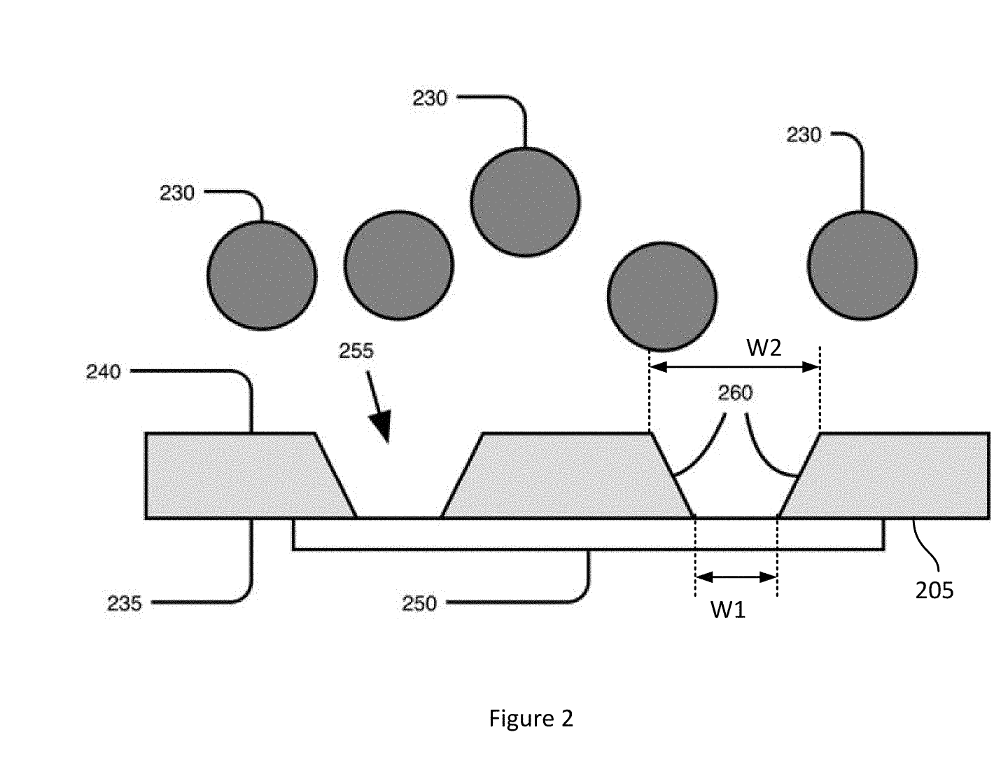

FIG. 2 depicts an example of a technique for generating a chassis that includes a plurality of electrodes, in accordance with various embodiments. Specifically, FIG. 2 depicts a simplified cross-sectional view of a number of vias 255 formed in a chassis 205. The chassis 205 may be similar to, for example, chassis 105. The chassis 205 may include an external surface 235 which may be similar to external surface 135. The chassis 205 may further include an internal surface 240 which may be opposite the external surface 235.

The vias 255 may be formed through one or more techniques such as mechanical drilling, chemical etching, optical etching, litho-etching, etc. In other embodiments where the chassis 205 is molded, for example injection-molded, the vias may be formed as part of the injection molding process during the formation of the chassis 205. Generally, in embodiments where the electrodes 130 are microspheres, the vias 255 may have a generally circular shape wherein the faces 260 of the vias 255 are sloped. More specifically, the vias 255 may have a width W1 at the external surface 235 with a smaller diameter than a width W2 at the internal surface. Generally, the width W2 may be larger than a diameter of electrodes 230 (which may be similar to electrodes 130). The width W1 may be smaller than a diameter of electrodes 230. As one example, in an embodiment where the electrodes have a diameter of approximately 0.8 mm, the width W2 may be approximately 1.65 mm and the width W1 may be approximately 0.58 mm.

Subsequent to, or concurrent with, the formation of vias 255 a mask 250 may be positioned on the external surface 235 of the chassis 205. The mask 250 may be formed of a material such as a plastic material, stainless steel, or some other material that may be generally non-reactive. The mask 250 itself may include a weak non-residue adhesive or a conductive adhesive material on a surface of the mask 250 that faces the external surface 235 of the chassis 205. The adhesive material may be a light adhesive, a solder flux, or some other material. More generally, the mask 250 may be formed of a tacky substance that is either conductive, or a non-residue type of adhesive so that the conductivity of the surface of the electrodes 230 is not negatively affected by contact with the mask 250. Generally, the adhesive material may provide two purposes. A first purpose may be to adhere the mask 250 to the external surface 235 of the chassis. The second purpose may be to provide a relatively “sticky” surface in the vias 255. A number of electrodes 230 may then be deposited over the internal surface 240 of the chassis 205. Electrodes 230 that enter the vias 255 may encounter the slightly sticky mask 250, which may hold the electrodes 230 in place within the vias 255.

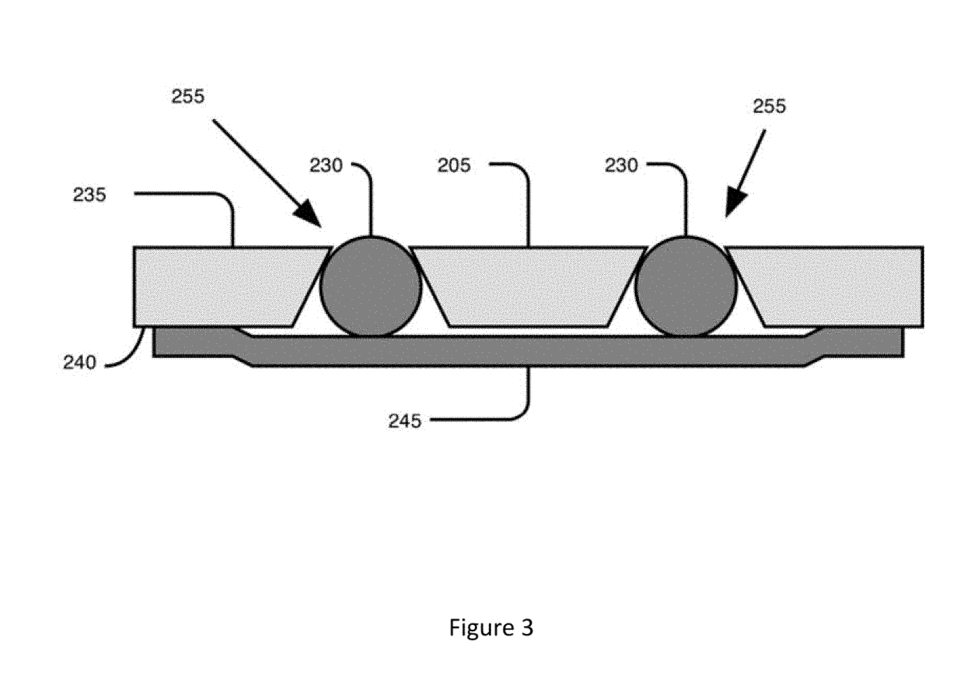

FIG. 3 depicts an example of the electrodes 230 being held in place with the vias 255. The mask 250 may be removed and an electrical interconnect 245 may be coupled to the internal surface 240 of the chassis 205. The electrical interconnect 245 may be coupled with the chassis 205 by an adhesive material, a solder reflow, rivets, screws, latches, or some other permanent or non-permanent coupling technique. The chassis 205 may then be flipped over as shown in FIG. 3 such that the external side 235 is “up.” The chassis 205 may then be coupled with one or more additional elements to form a computing device such as computing device 100.

In embodiments, the electrical interconnect 245 may include one or more of a conductive adhesive, metallic plates, rigid printed circuits, flexible printed circuits, metallized plastic, polyethylene terephthalate (PET) sheets, or some other material. In embodiments, the electrical interconnect 245 may serve a variety of purposes. First, the electrical interconnect 245 may exert a pressure against the electrodes 230 which may help hold the electrodes 230 within the via 255, particularly when a user exerts a pressure against the external surface 235 of the chassis 205. In addition, the electrical interconnect 245 may include one or more pads, vias, traces, etc. that may allow a signal to be conveyed between an electrode 230 and a processor of the computing device to which the chassis 205 is coupled. Specifically, the electrode 230 may generate one or more signals based on contact with a skin of a user. The signals may be transmitted from the electrode 230, through the electrical interconnect 245, to a processor of the computing device. The processor may be configured to perform one or more of the functions described above such as multiplexing the signal(s), processing the signals to generate a measurement related to a biosignal, etc.

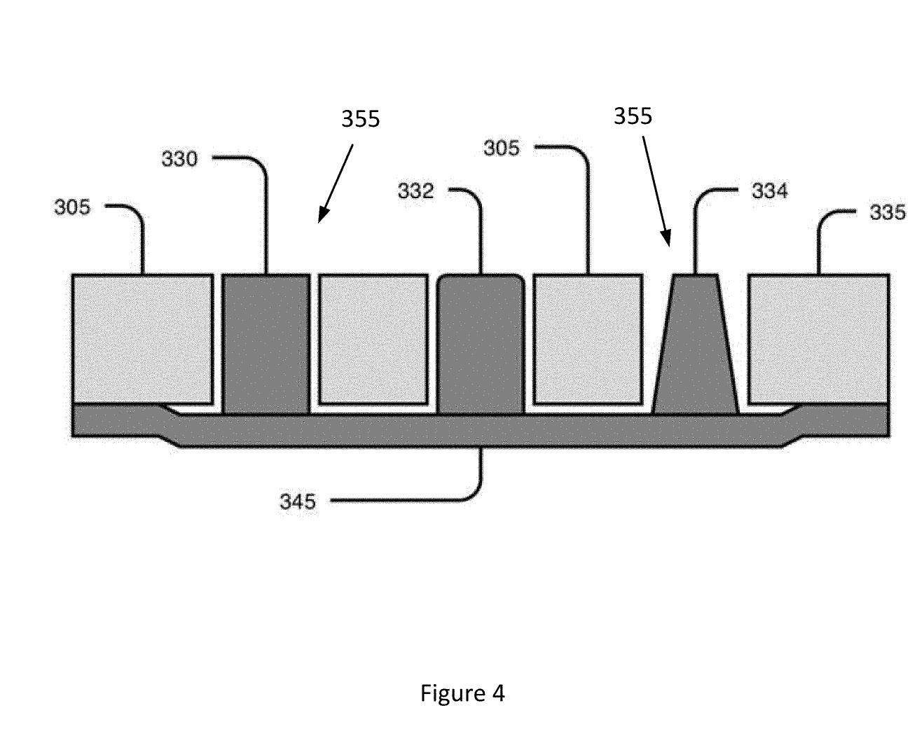

FIG. 4 depicts an alternative embodiment of a chassis 305, which may be similar to chassis 205. Specifically, FIG. 4 depicts a simplified cross-sectional view of the chassis 305 and a number of electrodes. The chassis 305 may be coupled with an electrical interconnect 345, which may be similar to electrical interconnect 245.

In embodiments, the electrical interconnect 345 may be coupled with a number of electrodes 330, 332, or 334. Specifically, the chassis 305 may include a number of vias 355, which may be formed in a fashion similar to vias 255. The vias 355 may have a number of electrodes 330, 332, or 334 positioned therein. The electrodes may be configured to perform a signal-generation function similar to that of electrodes 130 or 230 described above, but may have a different shape. Specifically, in some embodiments, one or more of the electrodes may have a profile similar to that of electrode 330 in that the electrodes may be generally squared off. In some embodiments, one or more of the electrodes such as electrode 332 may have a generally rounded-off shape. In some embodiments, one or more of the electrodes such as electrode 334 may have a “pin” type shape wherein a portion of the electrode 334 is narrower than another portion of the electrode 334. In some embodiments, the tops of one or more of the electrodes such as electrodes 330, 332, or 334 may be generally flush with the external surface 335 of the chassis 305.

In some embodiments, one or more of the electrodes 330, 332, or 334 may be positioned in a via 355 of the chassis prior to coupling the electrical interconnect 345 to the chassis 305. When the electrical interconnect 345 is coupled to the chassis 305, the electrode that is already present in the via 355 may be both physically and communicatively coupled with the electrical interconnect 345. In other embodiments, one or more of the electrodes 330, 332, or 334 may be coupled with the electrical interconnect 345. The electrodes may be inserted within the via 355 of the chassis 305 as part of coupling the electrical interconnect 345 with the chassis 305. In other embodiments, one or more of the electrodes 330, 332, or 334 may be placed within the vias 355 in accordance with some other technique.

It will be understood that this embodiment is intended as an example to depict variations that may be present in various embodiments. In some embodiments computing devices may include some combination of one or more of electrodes 230, 330, 332, and 334. In some embodiments one or more of the electrodes may be flush with the external surface of the chassis such as the electrodes 330, 332, and 334 shown in FIG. 4, while others of the electrodes may protrude from the external surface of the chassis such as the electrodes 230 shown in FIG. 3. In some embodiments one or more of the vias may have relatively straight faces such as is shown in FIG. 4, sloped faces as shown in FIG. 3, or combinations thereof.

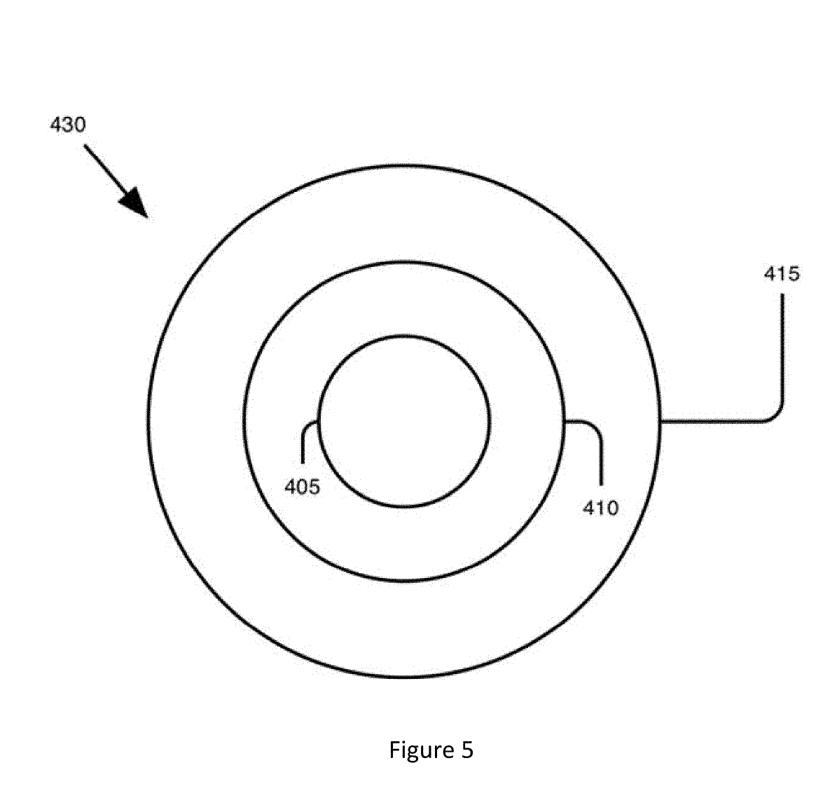

FIG. 5 depicts a cross-sectional view of an electrode 430, which may be similar to electrode 230. Specifically, the electrode 430 may be a microsphere that includes a core 405, an inner layer 410, and an outer layer 415. In embodiments the core 405 may include stainless steel, copper, brass, aluminum, zinc, metal alloys, or some other material that may provide a solid core for the electrode 430. The inner layer 410, which may also be referred to as a coating or plating layer, may be to ensure that the core material 405 may be electrically coupled with, for example, an electrical interconnect such as electrical interconnect 245 or 345. The inner layer 410 may be, for example, nickel, tin, copper, gold, or some other electrically conductive material. The outer layer 415 may also be referred to as a coating or plating layer. The outer layer 415 may be, for example, a surface coat layer such as black satin chrome or some other type of surface coat layer. In embodiments, the outer layer 415 may be styled to have a specific color or finish texture which may be dictated, for example, by the overall design aesthetic of the computing device in which the electrode 430 is to be used.

It will be understood that the above examples given with respect to FIG. 5 are intended as some examples, and other embodiments may have variations on the configuration of FIG. 5. For example, in some embodiments the outer layer 415 may not be present, or it may only be present in portions of the electrode 430 that are exposed to contact with a user of the computing device (e.g., portions of the electrode 430 that protrude from an external surface of a chassis or are otherwise exposed by the chassis). In some embodiments the inner layer 410 or the outer layer 415 may be composed of a plurality of layers. In some embodiments, one or more of the materials used in the core 405, the inner layer 410, or the outer layer 415 may be selected at least partially based on the biosignal that the electrode 430 is intended to monitor. In other words, the material used for one or more of the core 405, the inner layer 410, or the outer layer 415 may be different for an electrode that is intended to provide signals related to skin conductivity than for a biosensor that is intended to provide signals related to skin temperature. Additionally, it will be understood that although the various elements depicted in FIG. 5 are depicted with respect to a microsphere, other electrodes such as electrodes 430, 432, or 434 may similarly have a core material such as core material 405, surrounded by an inner layer such as inner layer 410, which in turn is surrounded by an outer layer such as outer layer 415.

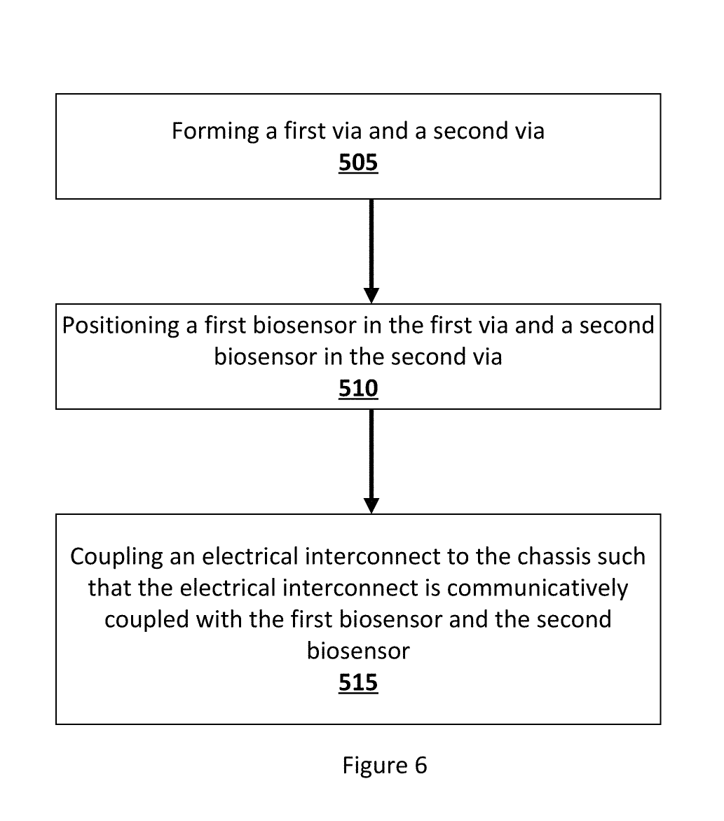

FIG. 6 depicts an example technique by which a chassis with one or more electrodes and an electrical interconnect may be formed. Specifically, the technique may include forming, at 505, a first via and a second via in a chassis. The first via and the second via may be similar to, for example, vias 255 or 355 that are respectively formed in chassis 205 or 305. As noted above, the vias may be formed at 505 using various techniques such as mechanical drilling, mechanical etching, chemical etching, optical etching, laser cutting, photolithography, injection molding, etc.

The technique may further include positioning, at 510, a first electrode in the first via and a second electrode in the second via. The electrodes may be, for example, similar to electrodes 230, 330, 332, 334, 430, etc. The electrodes may be positioned within the vias in accordance with the techniques discussed above with respect to FIGS. 2/3, or the embodiment depicted in FIG. 4. Specifically, in some embodiments a mask may be applied to the chassis and the electrodes may be deposited within the via where they adhere to the mask to stay in place. In other embodiments the electrodes may be coupled with an electrical interconnect and the electrodes is positioned within the via when the electrical interconnect is coupled with the chassis. In other embodiments the electrodes may be positioned within the vias in other ways.

The technique may further include coupling, at 515, an electrical interconnect to the chassis such that the electrical interconnect is communicatively coupled with the first biosensor and the second biosensor. The electrical interconnect may be similar to, for example, electrical interconnects 245 or 345.

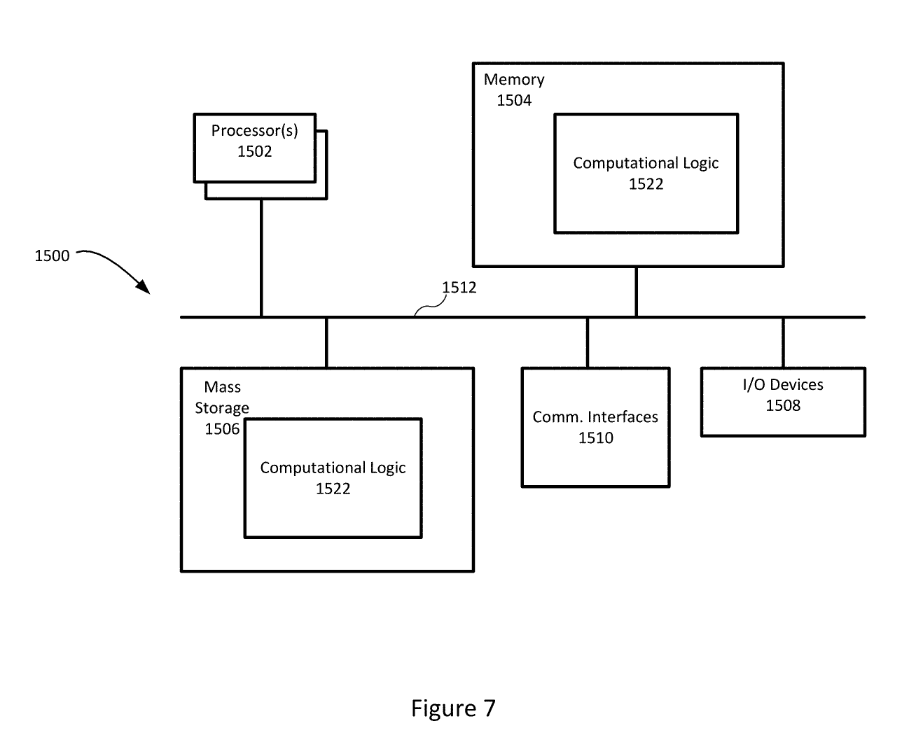

FIG. 7 illustrates an example computing device 1500 suitable for use with computing device 100 or chassis 205/305, in accordance with various embodiments. Specifically, in some embodiments, the computing device 1500 may be computing device 100, or may include a chassis such as chassis 205 or 305 with one or more electrodes positioned therein.

As shown, computing device 1500 may include one or more processors or processor cores 1502 and system memory 1504. For the purpose of this application, including the claims, the terms “processor” and “processor cores” may be considered synonymous, unless the context clearly requires otherwise. The processor 1502 may include any type of processors, such as a central processing unit (CPU), a microprocessor, and the like. The processor 1502 may be implemented as an integrated circuit having multi-cores, e.g., a multi-core microprocessor. The computing device 1500 may include mass storage devices 1506 (such as diskette, hard drive, volatile memory (e.g., dynamic random-access memory (DRAM), compact disc read-only memory (CD-ROM), digital versatile disk (DVD), and so forth). In general, system memory 1504 and/or mass storage devices 1506 may be temporal and/or persistent storage of any type, including, but not limited to, volatile and non-volatile memory, optical, magnetic, and/or solid state mass storage, and so forth. Volatile memory may include, but is not limited to, static and/or dynamic random-access memory. Non-volatile memory may include, but is not limited to, electrically erasable programmable read-only memory, phase change memory, resistive memory, and so forth.

The computing device 1500 may further include input/output (I/O) devices 1508 (such as a display (e.g., a touchscreen display), keyboard, cursor control, remote control, gaming controller, image capture device, and so forth) and communication interfaces 1510 (such as network interface cards, modems, infrared receivers, radio receivers (e.g., Bluetooth), and so forth). In some embodiments, the host device 103 may be elements of computing device 1500 such as processor(s) 1502, memory 1504, mass storage 1506, etc.

The communication interfaces 1510 may include communication chips (not shown) that may be configured to operate the device 1500 in accordance with a Global System for Mobile Communication (GSM), General Packet Radio Service (GPRS), Universal Mobile Telecommunications System (UMTS), High Speed Packet Access (HSPA), Evolved HSPA (E-HSPA), or Long-Term Evolution (LTE) network. The communication chips may also be configured to operate in accordance with Enhanced Data for GSM Evolution (EDGE), GSM EDGE Radio Access Network (GERAN), Universal Terrestrial Radio Access Network (UTRAN), or Evolved UTRAN (E-UTRAN). The communication chips may be configured to operate in accordance with Code Division Multiple Access (CDMA), Time Division Multiple Access (TDMA), Digital Enhanced Cordless Telecommunications (DECT), Evolution-Data Optimized (EV-DO), derivatives thereof, as well as any other wireless protocols that are designated as 3G, 4G, 5G, and beyond. The communication interfaces 1510 may operate in accordance with other wireless protocols in other embodiments.

The above-described computing device 1500 elements may be coupled to each other via system bus 1512, which may represent one or more buses. In the case of multiple buses, they may be bridged by one or more bus bridges (not shown). Each of these elements may perform its conventional functions known in the art. The various elements may be implemented by assembler instructions supported by processor(s) 1502 or high-level languages that may be compiled into such instructions.

The permanent copy of the programming instructions may be placed into mass storage devices 1506 in the factory, or in the field, through, for example, a distribution medium (not shown), such as a compact disc (CD), or through communication interface 1510 (from a distribution server (not shown)). That is, one or more distribution media having an implementation of the agent program may be employed to distribute the agent and to program various computing devices.

The number, capability, and/or capacity of the elements 1508, 1510, 1512 may vary, depending on whether computing device 1500 is used as a stationary computing device, such as a set-top box or desktop computer, or a mobile computing device, such as a tablet computing device, laptop computer, game console, or smartphone. Their constitutions are otherwise known, and accordingly will not be further described.

For one embodiment, at least one of processors 1502 may be packaged together with computational logic 1522 configured to practice aspects of optical signal transmission and receipt described herein to form a System in Package (SiP) or a System on Chip (SoC).

In various implementations, the computing device 1500 may comprise one or more components of a data center, a laptop, a netbook, a notebook, an ultrabook, a smartphone, a tablet, a personal digital assistant (PDA), an ultra mobile PC, a mobile phone, or a digital camera. In further implementations, the computing device 1500 may be any other electronic device that processes data.

In embodiments, the electrodes such as electrodes 130, 230, 330, etc. may be coupled with a processor 1502, which may be configured to receive one or more signals from the electrodes and perform one or more processing operations on the signals. The processing operations may include, for example, multiplexing the signals, analyzing the signals to identify a measurement related to a biosignal, or some other process. More specifically, the electrical interconnects 245 or 345 may be communicatively coupled with the bus 1512 to allow communication between the various electrodes and the processor 1502.

### Examples of Various Embodiments

Example 1 includes a computing device comprising: a chassis with an external surface that is accessible by a user of the computing device, and an internal surface opposite the external surface; a first electrode within the external surface, wherein the first electrode is to generate a first signal related to a first biosignal of the user; and a second electrode within the external surface, wherein the second electrode is to generate, concurrently with the generation of the first signal, a second signal related to a second biosignal of the user.

Example 2 includes the computing device of example 1, wherein the first electrode protrudes from the external surface by between 0 and 150 micrometers.

Example 3 includes the computing device of example 1, wherein the first biosignal is different from the second biosignal.

Example 4 includes the computing device of example 1, wherein the first biosignal is a skin temperature of the user, a skin conductance of the user, a heart-rate of the user, electromyography (EMG) of the user, or electroencephalogram (EEG) of the user.

Example 5 includes the computing device of example 1, wherein the first electrode is a pillar; and wherein the pillar has an exposed surface area at the external surface of the chassis of approximately 20 square millimeters (mm²).

Example 6 includes the computing device of example 1, wherein the first electrode is spherical.

Example 7 includes the computing device of example 6, wherein the first electrode has a diameter of less than 0.5 millimeters.

Example 8 includes the computing device of example 6, wherein the first electrode includes a core material, a first plating layer that surrounds the core material, and a second plating layer that at least partially surrounds the first plating layer.

Example 9 includes the computing device of example 8, wherein the core material includes stainless steel, copper, brass, zinc, aluminum, or magnesium.

Example 10 includes the computing device of example 8, wherein the first plating layer includes nickel.

Example 11 includes the computing device of example 8, wherein the second plating layer includes black satin chrome.

Example 12 includes the computing device of any of examples 1-11, wherein the first electrode and the second electrode are electrodes of a plurality of electrodes.

Example 13 includes the computing device of example 12, wherein the plurality of electrodes includes between 20 and 30 electrodes.

Example 14 includes the computing device of any of examples 1-11, wherein a center of the first electrode is between 2 millimeters (mm) and 5 mm from a center of the second electrode in a direction parallel to the external surface.

Example 15 includes the computing device of any of examples 1-11, wherein the first electrode and the second electrode are simultaneously contactable by a hand of the user.

Example 16 includes the computing device of any of examples 1-11, further comprising an electrical interconnect adjacent to the internal surface, wherein the electrical interconnect is physically and communicatively coupled with the first electrode and the second electrode.

Example 17 includes the computing device of any of examples 1-11, wherein the chassis includes a via between the external surface and the internal surface, wherein the via is sloped such that it is wider at the internal surface than the external surface, and wherein the first electrode is positioned within the via.

Example 18 includes the computing device of any of examples 1-11, wherein the first electrode is further to generate a third signal related to a third biosignal of the user.

Example 19 includes the computing device of example 18, further comprising a processor coupled with the first sensor, the processor to: time multiplex the first signal and the third signal; identify, based on the time multiplexed first signal, a measurement related to the first biosignal; and identify, based on the time multiplexed third signal, a measurement related to the third biosignal.

Example 20 includes the computing device of any of examples 1-11, further comprising a processor communicatively coupled with the first electrode, the processor to identify, based on the first signal related to the first biosignal of the user, a measurement related to the first biosignal.

Example 21 includes the computing device of example 20, further comprising a processor communicatively coupled with the first electrode, wherein the processor is to identify the measurement based on an identification that the first signal is a result of user contact with the first electrode.

Example 22 includes a computing device capable of simultaneously identifying a first measurement related to a first biosignal of a user and a second measurement related to a second biosignal of the user, wherein the computing device comprises: a chassis with an internal surface and an external surface opposite the internal surface; a first biosensor positioned within the chassis, wherein the first biosensor is exposed by the external surface of the chassis; a second biosensor positioned with the chassis, wherein the second biosensor is exposed by the external surface of the chassis; an electronic interconnect communicatively and physically coupled with the first biosensor and the second biosensor; and a processor communicatively coupled with the electronic interconnect, wherein the processor is to: identify, based on a first signal received from the first biosensor, a measurement related to a first biosignal of the user; and identify, based on a second signal received from the second biosensor, a measurement related to a second biosignal of the user.

Example 23 includes the computing device of example 22, wherein the first biosensor protrudes from the external side of the chassis by between 0 and 150 micrometers.

Example 24 includes the computing device of example 22, wherein the first biosignal is different from the second biosignal.

Example 25 includes the computing device of example 22, wherein the first biosignal is a skin temperature of the user, a skin conductance of the user, a heart-rate of the user, electromyography (EMG) of the user, or electroencephalogram (EEG) of the user.

Example 26 includes the computing device of example 22, wherein the first biosensor is a pillar; and wherein the pillar has an exposed surface area at the external surface of the chassis of approximately 20 square millimeters (mm²).

Example 27 includes the computing device of example 22, wherein the first biosensor is spherical.

Example 28 includes the computing device of example 27, wherein the first biosensor has a diameter of less than 0.5 millimeters.

Example 29 includes the computing device of example 27, wherein the first biosensor includes a core material, a first plating layer that surrounds the core material, and a second plating layer that at least partially surrounds the first plating layer.

Example 30 includes the computing device of example 29, wherein the core material includes stainless steel, copper, brass, zinc, aluminum, or magnesium.

Example 31 includes the computing device of example 29, wherein the first plating layer includes nickel.

Example 32 includes the computing device of example 29, wherein the second plating layer includes black satin chrome.

Example 33 includes the computing device of any of examples 22-32, wherein the first biosensor and the second biosensor are biosensors of a plurality of biosensors.

Example 34 includes the computing device of example 33, wherein the plurality of biosensors includes between 20 and 30 biosensors.

Example 35 includes the computing device of any of examples 22-32, wherein a center of the first biosensor is between 2 millimeters (mm) and 5 mm from a center of the second biosensor in a direction parallel to the external surface.

Example 36 includes the computing device of any of examples 22-32, wherein the first biosensor and the second biosensor are simultaneously contactable by a hand of the user.

Example 37 includes the computing device of any of examples 22-32, further comprising an electrical interconnect adjacent to the internal surface, wherein the electrical interconnect is physically and communicatively coupled with the first biosensor and the second biosensor.

Example 38 includes the computing device of any of examples 22-32, wherein the chassis includes a via between the external surface and the internal surface, wherein the via is sloped such that it is wider at the internal surface than the external surface, and wherein the first biosensor is positioned within the via.

Example 39 includes the computing device of any of examples 22-32, wherein the first biosensor is further to generate a third signal related to a third biosignal of the user.

Example 40 includes the computing device of example 39, wherein the processor is further to: time multiplex the first signal and the third signal; identify, based on the time multiplexed first signal, the measurement related to the first biosignal; and identify, based on the time multiplexed third signal, a measurement related to the third biosignal.

Example 41 includes the computing device of any of examples 22-32, wherein the processor is to identify the measurement related to the first biosignal of the user based on an identification that the first signal is a result of user contact with the first biosensor.

Example 42 includes a method of manufacturing a chassis that includes a first biosensor and a second biosensor, wherein the method comprises: forming, between a first side of the chassis and a second side opposite the first side, a first via and a second via; positioning a first biosensor in the first via and a second biosensor in the second via wherein the first biosensor and the second biosensor are exposed at the first side of the chassis; and coupling an electrical interconnect with the second side of the chassis such that the electrical interconnect is communicatively coupled with the first biosensor and the second biosensor.

Example 43 includes the method of example 42, wherein forming the first via and the second via includes forming the first via and the second via during formation of the chassis.

Example 44 includes the method of example 42, wherein the first via has faces that are sloped.

Example 45 includes the method of example 42, wherein the first biosensor is a pillar.

Example 46 includes the method of example 42, wherein the first biosensor is spherical.

Example 47 includes the method of any of examples 42-46, wherein positioning the first biosensor in the first via includes coupling the first biosensor to a mask attached to the first side of the chassis.

Example 48 includes the method of any of examples 42-46, wherein the first biosensor is communicatively and physically coupled with the electrical interconnect prior to positioning the first biosensor in the first via, and positioning the first biosensor in the first via includes coupling the electrical interconnect with the second side of the chassis.

Example 49 includes one or more non-transitory computer-readable media comprising instructions that, upon execution of the instructions by a processor of a computing device, are to cause the processor to: identify, based on a first signal received from a first electrode positioned in a chassis of the computing device, a measurement related to a first biosignal of the user; and identify, based on a second signal received from the second electrode positioned in the chassis of the computing device, a measurement related to a second biosignal of the user; wherein the first signal and the second signal are generated concurrently with one another based on contact with the skin of the user.

Example 50 includes the one or more non-transitory computer-readable media of example 49, wherein the processor is further to: identify a third signal related to a third biosignal of the user; time multiplex the first signal and the third signal; identify, based on the time multiplexed first signal, the measurement related to the first biosignal of the user; and identifying, based on the time multiplexed third signal, the measurement related to the third biosignal of the user.

Example 51 includes the one or more non-transitory computer-readable media of example 50, wherein the third signal is generated by the first biosignal.

Example 52 includes the one or more non-transitory computer-readable media of example 49, wherein the instructions are further to identify that the first signal is based on user contact with the first electrode.

Example 53 includes the one or more non-transitory computer-readable media of any of examples 49-52, wherein the first biosignal is a skin temperature of the user, a skin conductance of the user, a heart-rate of the user, electromyography (EMG) of the user, or electroencephalogram (EEG) of the user.

Various embodiments may include any suitable combination of the above-described embodiments including alternative (or) embodiments of embodiments that are described in conjunctive form (and) above (e.g., the “and” may be “and/or”). Furthermore, some embodiments may include one or more articles of manufacture (e.g., non-transitory computer-readable media) having instructions, stored thereon, that when executed result in actions of any of the above-described embodiments. Moreover, some embodiments may include apparatuses or systems having any suitable means for carrying out the various operations of the above-described embodiments.

The above description of illustrated implementations of the various embodiments, including what is described in the Abstract, is not intended to be exhaustive or to limit the invention to the precise forms disclosed. While specific implementations of, and examples for, various embodiments are described herein for illustrative purposes, various equivalent modifications are possible within the scope of the invention, as those skilled in the relevant art will recognize. These modifications may be made in light of the above detailed description. The terms used in the following claims should not be construed to limit this disclosure to the specific embodiments disclosed in the specification and the claims.

1. A computing device comprising:
a chassis with an external surface that is accessible by a user of the computing device, and an internal surface opposite the external surface;
a first electrode within the external surface, wherein the first electrode is to generate a first signal related to a first biosignal of the user; and
a second electrode within the external surface, wherein the second electrode is to generate, concurrently with the generation of the first signal, a second signal related to a second biosignal of the user.

2. The computing device of claim 1, wherein the first biosignal is different from the second biosignal.

3. The computing device of claim 1, wherein the first biosignal is a skin temperature of the user, a skin conductance of the user, a heart-rate of the user, electromyography (EMG) of the user, or electroencephalogram (EEG) of the user.

4. The computing device of claim 1, wherein the first electrode is a pillar; and
wherein the pillar has an exposed surface area at the external surface of the chassis of approximately 20 square millimeters (mm2).

5. The computing device of claim 1, wherein the first electrode is spherical.

6. A computing device capable of simultaneously identifying a first measurement related to a first biosignal of a user and a second measurement related to a second biosignal of the user, wherein the computing device comprises:
a chassis with an internal surface and an external surface opposite the internal surface;
a first biosensor positioned within the chassis, wherein the first biosensor is exposed by the external surface of the chassis;
a second biosensor positioned with the chassis, wherein the second biosensor is exposed by the external surface of the chassis;
an electronic interconnect communicatively and physically coupled with the first biosensor and the second biosensor; and
a processor communicatively coupled with the electronic interconnect, wherein the processor is to:
identify, based on a first signal received from the first biosensor, a measurement related to a first biosignal of the user; and
identify, based on a second signal received from the second biosensor, a measurement related to a second biosignal of the user.

7. The computing device of claim 6, wherein the first biosensor is a pillar; and
wherein the pillar has an exposed surface area at the external surface of the chassis of approximately 20 square millimeters (mm2).

8. The computing device of claim 6, wherein the first biosensor is spherical.

9. The computing device of claim 6, wherein the first biosensor and the second biosensor are biosensors of a plurality of biosensors.

10. The computing device of claim 6, wherein a center of the first biosensor is between 2 millimeters (mm) and 5 mm from a center of the second biosensor in a direction parallel to the external surface.

11. The computing device of claim 6, wherein the first biosensor is further to generate a third signal related to a third biosignal of the user.

12. The computing device of claim 11, wherein the processor is further to:
time multiplex the first signal and the third signal;
identify, based on the time multiplexed first signal, the measurement related to the first biosignal; and
identify, based on the time multiplexed third signal, a measurement related to the third biosignal.

13. The computing device of claim 6, wherein the processor is to identify the measurement related to the first biosignal of the user based on an identification that the first signal is a result of user contact with the first biosensor.

14. A method of manufacturing a chassis that includes a first biosensor and a second biosensor, wherein the method comprises:
forming, between a first side of the chassis and a second side opposite the first side, a first via and a second via;
positioning a first biosensor in the first via and a second biosensor in the second via wherein the first biosensor and the second biosensor are exposed at the first side of the chassis; and
coupling an electrical interconnect with the second side of the chassis such that the electrical interconnect is communicatively coupled with the first biosensor and the second biosensor.

15. The method of claim 14, wherein forming the first via and the second via includes forming the first via and the second via during formation of the chassis.

16. The method of claim 14, wherein the first via has faces that are sloped.

17. The method of claim 14, wherein the first biosensor is a pillar.

18. The method of claim 14, wherein the first biosensor is spherical.

19. The method of claim 14, wherein positioning the first biosensor in the first via includes coupling the first biosensor to a mask attached to the first side of the chassis.

20. The method of claim 14, wherein the first biosensor is communicatively and physically coupled with the electrical interconnect prior to positioning the first biosensor in the first via, and positioning the first biosensor in the first via includes coupling the electrical interconnect with the second side of the chassis.

21. One or more non-transitory computer-readable media comprising instructions that, upon execution of the instructions by a processor of a computing device, are to cause the processor to:
identify, based on a first signal received from a first electrode positioned in a chassis of the computing device, a measurement related to a first biosignal of a user; and
identify, based on a second signal received from a second electrode positioned in the chassis of the computing device, a measurement related to a second biosignal of the user;
wherein the first signal and the second signal are generated concurrently with one another based on contact with skin of the user.

22. The one or more non-transitory computer-readable media of claim 21, wherein the processor is further to:
identify a third signal related to a third biosignal of the user;
time multiplex the first signal and the third signal;
identify, based on the time multiplexed first signal, the measurement related to the first biosignal of the user; and
identifying, based on the time multiplexed third signal, the measurement related to the third biosignal of the user.

23. The one or more non-transitory computer-readable media of claim 22, wherein the third signal is generated by the first biosignal.

24. The one or more non-transitory computer-readable media of claim 21, wherein the instructions are further to identify that the first signal is based on user contact with the first electrode.

25. The one or more non-transitory computer-readable media of claim 21, wherein the first biosignal is a skin temperature of the user, a skin conductance of the user, a heart-rate of the user, electromyography (EMG) of the user, or electroencephalogram (EEG) of the user.
